(19) United States
(12) Patent Application Publication
(10) Pub. No.: US 2007/0154495 A1
(43) Pub. Date: Jul. 5, 2007

(54) Modified whole cell, cell extract and omv-based vaccines

(75) Inventors: Andrew R. Gorringe, Salisbury (GB); Karen M. Reddin, Salisbury (GB); Scott D. Gray-Owen, Oakville (CA); Ian C. Boulton, Toronto (CA)

Correspondence Address: HODGSON RUSS LLP;THE GUARANTY BUILDING, 140 PEARL STREET, SUITE 100, BUFFALO, NY, 14202-4040, US

(73) Assignee: Health Protection Agency, Salisbury, Wiltshire (GB)

(21) Appl. No.: 10/575,070

(22) PCT Filed: Oct. 8, 2004
(86) PCT No.: PCT/GB04/04274

§ 371 Date: Jan. 29, 2007

(30) Foreign Application Priority Data
Oct. 9, 2003 (GB) 0323709.6

(51) Int. Cl. A61K 39/095; C12N 1/21

(52) U.S. Cl. 424/249.1; 424/250.1; 435/252.3

(57) ABSTRACT

Live vaccines, live-attenuated vaccines, dead vaccines, protein preparations for use in vaccines and OMVs for use in vaccines are free of CEACAM1-binding Opa, though max contain Opa variants that are antigenic but don't bind to CEACAM1.

The invention relates to modified whole cell, cell extract and OMV-based compositions and components thereof for treatment or prevention of disease by Gram negative bacteria, in particular disease caused by Neisseria.

A significant number of human and animal pathogens fall within the Gram negative classification of bacteria, including members of the genera Neisseria, Moraxella, Kingella, Acinetobacter, Brucella, Bordetella, Haemophilus, Escherichia, Chlamydia, Legionella, Pseudomonas, Proteus and Yersinia. Neisseria meningitidis (the meningococcus) is the organism that causes meningococcal meningitis and is of particular importance as a worldwide health problem. In many countries the incidence of this disease is increasing. N. meningitidis is also responsible for meningococcal septicaemia, which is associated with rapid onset of symptoms and high mortality, with around 22% of cases proving fatal. Other Gram negative bacteria cause a range of human infections including meningitis (H. influenzae), plague (Y. pestis), gastroenteritis (E. coli), venereal disease (N. gonorrhoeae) and nosocomial infection (P. aeruginosa).

It is desirable to provide broad spectrum vaccines that provide protective immunity in animals, particularly humans, against Gram negative bacterial infection.

Outer membrane vesicles (OMVs) derived from the human pathogen Neisseria meningitidis are currently utilized as a source of antigen for a protective meningococcal vaccine.

To address the difficulties associated with achieving broad spectrum protection researchers have attempted to “enrich” OMVs with particular antigens that might enhance the immunogenic potential of the OMV. In WO-A-00/25811 OMvs isolated from N. meningitidis are combined with heterologous antigens, e.g. Transferrin binding protein (Thp), or a genetically modified N. meningitidis expresses such antigens recombinantly and antigen enriched OMVs are derived therefrom. A similar approach was adopted in WO-A-01/09350 which describes vaccine compositions comprising OMVs from N. meningitidis, M. catarrhalis and H. influenzae. In certain embodiments these organisms have been genetically modified to overexpress particular immunogenic moieties.

A difficulty with such OMV-based vaccines is that, to achieve adequate protection, vaccines have to be administered at frequent intervals, or boosters have to be given to maintain a protective immune response.

Despite the availability of effective antibiotic therapies to combat infection, Neisseria gonorrhoeae causes about 78 million infections globally per annum. Gonorrhoea is characterized by an intense inflammatory response that leads to the liberation of large amounts of urethral or cervical pus, consisting primarily of neutrophils with extracellular and intracellular-associated N. gonorrhoeae. Up to 15% of infected men and 80% of infected women remain asymptomatic. In such situations, infection tends to be prolonged and is consistently transmissible, both vertically (to neonates of infected mothers) and horizontally (to sexual partners). If undetected, such infections are a source of significant morbidity, including severe conjunctivitis in neonates, disseminated gonococcal infection, pelvic inflammatory disease and sterility through fallopian tube scarring.

The persistence of N. gonorrhoeae within the population relies on the fact that gonorrhoea can be contracted repeatedly, and there is little evidence that the exposure to or colonization by this organism reduces an individual's susceptibility to subsequent infection. This is at least partially attributable to the antigenic variation of gonococcal surface epitopes, however, individuals can be reinfected by the same serotype of N. gonorrhoeae indicating that immune evasion is not the only survival strategy used by this pathogen.

It is hence desired to provide improved vaccination against initial or repeat gonococcal infection.

The colony Opacity (Opa) proteins expressed by Neisserial species are an important virulence determinant. Boulton et al: Nat Immunol. 2002 March; 3(3):229-36 describe how, in vitro, Opa proteins suppressed T lymphocyte activation and proliferation. Opa proteins are described across the literature as ideal vaccine targets. Wiertz et al (Infect Immun. 1996 January; 64(1):298-304.) and De Jonge et al (Infect Immun. 2003 May; 71(5):2331-40.) identify Opa-antibodies as important in protection against gonococcal infection. De Jonge et al noted how Opa-antibodies reduced Neisserial adhesion and so propose including Opa in vaccination. Schneider et al J Infect Dis. 1995 July; 172(1):180-5.) propose use of an Opa-based vaccine against gonorrhoea. Plummer et al (J. Clin. Invest. 1994; 93: 1748-1755) correlated antibodies to Opa with reduced risk of gonococcal salpingitis and promote Opa-based vaccines.

An object of the present invention is to provide microorganisms, compositions and vaccines, including OMV-based vaccines for use in treatment or prevention of disease by Gram negative bacteria. An object of specific objects of the invention is to provide such a vaccine that solves or at least ameliorates problems associated with current vaccination against meningococcal disease and gonorrhoea.

Accordingly, the invention provides microorganisms, compositions, vaccines, components of vaccines, methods of obtaining the aforementioned and genes encoding the aforementioned, substantially free of Opa that binds CEACAM1. These are suitable for use in treatment or prevention of disease caused by Gram negative bacteria. The invention also provides microorganisms, compositions, vaccines, components of vaccines, methods of obtaining the aforementioned and genes encoding the aforementioned that are suitable for treatment or prevention of meningococcal disease or gonococcal disease and are substantially free of protein that suppresses activation or proliferation of a CD4+ T cell.

The microorganisms are typically Gram negative bacteria, especially Neisseria, which are selected to be substantially free of Opa that binds CEACAM1 or that are modified so as to be substantially free of Opa that binds CEACAM1. They may be modified by mutation to be Opa-free or to express an Opa that does not bind CEACAM1. Compositions of the invention contain such bacteria or immunogenic extracts thereof, for example protein preparations thereof. Vaccines comprising the microorganisms or extracts thereof may contain live bacteria, live attenuated bacteria or dead bacteria. Generally, hereafter, reference to compositions of the invention is intended to refer to all of microorganisms, compositions, vaccines, and components of vaccines unless otherwise indicated.

A method of selecting a microorganism, composition, vaccine or vaccine component, for use in treatment or prevention of disease caused by Gram negative bacteria is in addition provided herein, the method comprising determining whether said microorganism, composition, vaccine or vaccine component is substantially free of Opa that binds CEACAM1.

A further method of the invention is one for selecting a microorganism, composition, vaccine or vaccine component, for use in treatment or prevention of meningococcal disease or gonococcal disease, comprising determining whether said microorganism, composition, vaccine or vaccine component is substantially free of protein that suppresses activation or proliferation of a CD4+ T cell.

Said microorganism, composition, vaccine or vaccine component is preferably substantially free of Opa that binds CEACAM1 or is modified so as to be substantially free of Opa that binds CEACAM1. In a preferred method a Neisseria is selected to be substantially free of Opa that binds CEACAM1 or is modified, such as by mutation as described in more detail below, so as to be substantially free of Opa that binds CEACAM1.

A population of Gram negative bacteria of the invention, being 1,000 or more in number, is substantially free of bacteria expressing Opa that binds CEACAM1. Compositions are obtained therefrom which likewise are substantially free of Opa that binds CEACAM1.

In another embodiment, the present invention provides a composition, comprising Gram negative bacteria outer membrane vesicles, preferably Neisseria outer membrane vesicles, wherein the vesicles are substantially free of Opa.

The Opa content of the vesicles is preferably reduced by at least a factor of 10 compared with the Opa content of OMVs obtained from normal Neisseria, the benchmark for normal Neisseria being the Opa content of OMVs obtained from N. meningitidis strain K454, and more especially the Opa represents 0.5% or less by weight of the total protein content of OMVs.

A further composition of the invention comprises outer membrane vesicles, wherein the vesicles comprise an Opa protein that does not bind to CEACAM1. These vesicles may in addition be substantially free of Opa that binds CEACAM1.

A still further composition of the invention comprises outer membrane vesicles, wherein the vesicles comprise a protein which is antigenic, elicits production of antibodies which bind to Opa, and does not bind to CEACAM1. The protein may be a mutant or variant or fragment or derivative or mimic of Opa.

A yet further composition of the invention comprises outer membrane vesicles, wherein the vesicles comprise an antagonist which inhibits binding of Opa to CEACAM1.

The term “Opa” refers to a Gram negative, especially Neisserial, colony opacity associated protein that can modulate or suppress an immune response or inhibit tumor growth. Preferably, the Opa protein can bind to CEACAM1 and cause ligation of CEACAM1 with consequent immune suppression or inhibition of an immune cell.

Specific Opa proteins include Opa₅₂ and Opa₅₇. Since the neisserial Opa proteins are highly antigenically variable, the Opa protein may be any of the Opa proteins that can be expressed by various neisserial species and that also bind to the CEACAM1 receptor or to homologous non-human receptors. Opa proteins include the Opa proteins encoded by any neisserial species, including the pathogenic Neisseria gonorrhoeae and Neisseria meningitidis and the commensal species such as Neisseria lactamica and Neisseria subflava, for which their Opa proteins have been shown to bind CEACAM1, and other commensals that also express Opa proteins.

The term “Opa protein” also refers to analogous proteins from other bacterial species. This includes, but is not restricted to, the CEACAM1-binding proteins of Haemophilus influenzae. Like the Neisserial Opa proteins, the H. influenzae P5 proteins are antigenically variable outer membrane proteins that are predicted to form a beta-barrel structure with eight transmembrane regions and four extracellular loops. As with the Opa proteins, the P5 transmembrane regions and the 4th surface-exposed loop are well conserved, while the sequence within the other surface-exposed loops is variable. Also like various of the Neisserial Opa proteins, the H. influenzae P5 proteins function in attachment to host cells via binding to CEACAM receptors, including CEACAM1.

In the present invention, we provide compositions purposively lacking, or at least reduced in, CEACAM1-reactive Opa content. We also provide compositions containing mutants or variants or fragments or derivatives or mimics of Opa, which mutants, variants, fragments, mimics and derivatives do not activate CEACAM1. These compositions thus offer the basis of vaccines against disease, especially Neisserial disease, more especially meningococcal and gonococcal disease, with improved development of immune response and immune memory in patients. The mutants, variants, fragments and derivatives enable anti-Opa antibodies and other immune responses to be generated in vivo without the disadvantages of activation of CEACAM1, for example without immune suppression and/or reduction in immune memory.

Compositions of the invention are for use in treating patients, typically human patients, and the invention provides a method of treatment of an individual comprising administering a composition of the invention.

The invention also relates to manufacture of compositions for vaccination and to vaccine components. Accordingly, a method of the invention, for preparing a composition for use as or in manufacture of a vaccine, comprises:
(a) obtaining a Gram negative bacterium;
(b) determining whether the bacterium expresses an Opa protein that binds to CEACAM1;
(c) if the bacterium expresses the Opa protein, discarding the bacterium and repeating steps (a) to (c);
(d) retaining the bacterium if it does not express the Opa protein; and
(e) preparing a composition comprising the retained bacterium of step (d).

The bacterium is preferably a Neisseria.

The bacterium may be one that does not express CEACAM1-reactive Opa, but does produce non-functional Opa, and the method can include a further step of retaining a bacterium which expresses a mutant or variant or fragment or derivative or mimic of Opa, wherein the mutant or variant or fragment or derivative or mimic does not bind to CEACAM1.

Such mutants or variants or fragments or derivatives or mimics of Opa may arise naturally in the bacterial population. A preferred method of the invention, however, is one that induces production of such proteins, and comprises:
(a) obtaining a Gram negative bacterium;
(b) carrying out mutagenesis on the bacterium;
(c) determining whether the bacterium expresses a mutant or fragment or variant or derivative or mimic of an Opa protein that does not bind to CEACAM1;
(d) isolating the mutant or variant or fragment or derivative or mimic.

Neisseria species are naturally competent and amenable to mutagenesis via recombination between homologous DNA sequences and, further, the availability of the entire meningococcal and gonococcal genome sequences enable accurate determination of suitable sites for mutagenesis.

One method of creating or obtaining a bacterium of the invention is to clone an Opa gene, insert the cloned gene into an expression vector, and mutagenise the cloned gene. The cloned gene can then be expressed and its product tested for (i) binding to CEACAM1, and (ii) generating antibodies that bind to native Opa. A mutated gene which produces a product having desired properties can then be expressed in vitro to obtain a mutant protein of the invention. The protein can be incorporated into compositions, especially vaccines. This technique is also suitable for generating a fragment of Opa which likewise can be tested for the desired properties. The mutated gene can be inserted into a host bacterium, preferably a Neisseria, to replace a native CEACAM1 binding Opa.

Transposons (Tns) are widely used for mutagenesis where available. This is because 1) it is easy to map the site of mutation (insertion of the transposon) by sequencing out from the ends of the transposon; 2) transposons can be used which insert only once in the chromosome, allowing analysis of a mutant phenotype resulting from a single insertion mutation; 3) existing procedures allow simultaneous screening of large numbers of potentially interesting mutants. Other classical methods for mutagenesis include the use of UV light and, more frequently, the use of mutagenic agents to introduce physical changes in the DNA which results in the mutation of genes. The mutations introduced by such methods are far more random than those generated by Tn since individual base pairs are the target (typically G:C->A:T transitions). There is no direct requirement for complex genetic systems, as with Tn mutagenesis using these approaches, however vectors may be required to identify the site of mutation by complementation. Typically one establishes a dose vs. survival curve for the agent then uses the dose which kills approximately 90% of the population to ensure mutations are introduced. A more detailed protocol is given in the Examples for EMS and NTG mutagenesis.

It is desirable for the compositions of the invention, including OMV-based vaccines, to include antigens that will induce protective antibodies that bind to Opa in vivo. Hence, methods of the inventions for generating and identifying such antigens typically also include the steps of:
(e) raising an antibody to the mutant or fragment or variant or derivative; and
(f) determining whether the antibody also binds to an Opa protein that binds to CEACAM1.

Also provided by the present invention is an isolated mutant or variant or fragment or derivative or mimic of Opa, wherein the mutant or variant or fragment or derivative or mimic does not bind to CEACAM1, preferably as obtained according to the methods described herein.

OMV-based vaccines exist at present, using OMVs from various Neisserial species. Such vaccines may contain Opa protein that binds to CEACAM1, though the consequences of this have until now been unappreciated. In a further embodiment of the invention there is provided a method of manufacture or testing of a vaccine, the method comprising:
(a) obtaining a sample of a vaccine or of a component of a proposed vaccine against a Gram negative bacteria; and
(b) determining whether the sample contains an Opa protein that binds to CEACAM1.

Thus, it is now possible to screen new and existing vaccines to determine whether Opa content renders them suitable or unsuitable for therapeutic use. This information may also assist in explaining the different efficacies of respective vaccines in trials and commercial use.

A maximum level of Opa may be acceptable, and hence it is optional to determine the weight % of the Opa protein, if present, by weight % of total protein content in the vaccine or in the sample. The vaccine or the component may then be rejected if the sample contains the Opa protein, or if the weight % of the Opa protein is above a predetermined level, e.g. 0.5%.

The invention also provides use of (a) Neisseria or (b) Neisseria outer membrane vesicles which (i) are substantially free of Opa, (ii) comprise an Opa protein that does not bind to CEACAM1, (iii) comprise a mutant or variant or fragment or derivative of Opa that does not bind to CEACAM1, or (iv) comprise an antagonist which inhibits binding of Opa to CEACAM1, in manufacture of a medicament for treatment or prevention of meningococcal or gonococcal disease with improved stimulation of immune memory or reduced inhibition of T cell function (e.g. activation and/or proliferation).

The invention applies in particular to OMVs from Gram negative bacteria, being those bacteria that fail to resist decolourisation in the commonly known Gram staining method. Gram negative bacteria are characterised by a complex multilayer cell wall and often possess an outer layer polysaccharide capsule—e.g. N. meningitidis, although in some species this capsule is absent—e.g. N. lactamica.

Outer membrane vesicles (OMVs), also referred to as blebs, are vesicles formed or derived from fragments of the outer membrane of a Gram negative bacterium. OMvs typically comprise outermembrane proteins (OMPs), lipids, phospholipids, periplasmic material and lipopolysaccharide (LPS). Gram negative bacteria, especially pathogens like N. meningitidis, often shed OMVs during virulent infections in a process known as blebbing. OMVs can also be obtained from Gram negative bacteria via a number of known chemical denaturation processes. Liposomes, comprising a lipid bilayer and typically enclosing an aqueous core, can be regarded for the purposes of the present invention as constituting a synthetic equivalent to OMVs, and embodiments of the invention described with reference to OMVs apply mutatis mutandis to embodiments carried out with and relating to liposomes.

A “vaccine” as referred to herein is defined as a pharmaceutical or therapeutic composition used to inoculate an animal in order to immunize the animal against infection by an organism, typically a pathogenic organism. A vaccine will typically comprise one or more antigens derived from one or more organisms which on administration to an animal will stimulate active immunity and protect that animal against infection with these or related pathogenic organisms.

Vaccine compositions that are formulated as pharmaceuticals will typically comprise a carrier. If in solution or in liquid aerosol suspension, suitable carriers can include saline solution, sucrose solution, or other pharmaceutically acceptable buffer solutions. An aerosol formulation will typically additionally comprise a surfactant. Alternatively, vaccine compositions include microencapsulated OMV compositions. Such microcapsules will generally comprise a biocompatible polymer shell or core, such as made from polylactide-co-glycolide (PLG). Vaccine compositions can additionally comprise an adjuvant, for example where administration is via the parenteral route. Suitable adjuvants include aluminium hydroxide.

Vaccines are suitably administered to an animal via a number of routes. For example, parenterally—e.g. intramuscularly, trans-dermally—or via other routes—e.g. intra-nasally, orally, topically—or via any other commonly known administrative route.

Certain proteins can be recombinantly expressed in Gram negative bacteria and thereby enable enrichment or alteration of the antigenic profile of the bacterial outer membrane. Genetic modification of a bacterial source organism thereby allows for manipulation of the antigenic profile of OMVs that are obtained from these organisms. When proteins that are not normally present in the bacterial outer membrane, and thus in an OMV derived therefrom, are introduced via recombinant expression techniques, these “non-native” proteins and polypeptides are described as heterologous antigens. The contents of WO-A-00/25811 and WO-A-01/09350 are incorporated herein. Thus it is an advantage of embodiments of the invention that the vaccine comprises OMVs rather than live attenuated or dead pathogenic organisms which can pose a greater risk of infection or adverse reaction.

In general, all such bacteria are believed suitable, though Gram negative species especially suitable for use in the invention include those selected from Neisseria, Moraxella, Kingella, Acinetobacter, Brucella, Bordetella, Porphyromonas, Actinobacillus, Borelia, Serratia, Campylobacter, Helicobacter, Haemophilus, Escherichia, Legionella, Salmonella, Pseudomonas and Yersinia. In a particular embodiment of the invention the composition comprises OMVs from strains of Neisseria.

Suitable methods for extracting OMVs from bacterial sources include deoxycholate extraction, Tris/HCl/EDTA extraction, and lithium acetate extraction. Protocols for performing such extractions are described in more detail in the literature. However, it will be appreciated by the skilled person that virtually any chemical and/or physical technique that enables disruption of the bacterial cell outer membrane in order to release sufficient OMVs for purification and isolation, is suitable for preparation of the compositions of the invention.

Further aspects of the invention provide methods of vaccinating animals, especially humans, against Gram negative bacterial infection utilising the compositions of the invention. In particular, the invention provides methods for vaccinating animals against meningococcal infection. Also provided are uses of the compositions of the invention in the vaccination of animals, including humans, against Gram negative bacterial infection.

Further provided are uses of the compositions of the invention in the manufacture of vaccines for inoculating animals in order to stimulate protective immunity to Gram negative bacterial infection. OMVs are of use in mucosally administered compositions, as LPS toxicity is less and LPS can function as an adjuvant.

OMVs of the invention have reduced content of, or are free of, Opa proteins which recognize carcinoembryonic antigen-related cell adhesion molecule 1 (CEACAM1) and which suppress both the activation and proliferation of Jurkat CD4⁺ T lymphocytes in response to various stimuli in vitro and have similar effects on primary (non-malignant) cells of a similar type.

An advantage of the invention is that our OMVs avoid the presence of CEACAM1-reactive Opa and thereby, in comparison with OMVs that do contain CEACAM1-reactive Opa, show improved lymphocyte response to activating stimuli, decreased lymphocyte death, and by corollary, increased development of protective immunity. This is surprising as, given the species specificity of neisserial Opa proteins with regard to receptor recognition, these human immunosuppressive effects would not be evident in animal models. Hence, neisserial strains deficient in CEACAM1 binding activity are used in the development of OMVs-based human vaccines of the invention.

Pathogenic and commensal Neisseriacae are each capable of expressing Opa proteins which are, in the vast majority of instances, recognized by host CEACAM1. We have demonstrated the presence of such proteins in OMV preparations, and further, that these species are capable of interacting with human lymphocytes in a manner inducing immunosuppression.

We have noted significant differences in response to OMVs derived from N. meningitidis, (Nm) N. lactamica (Nl) and N. gonorrhoeae (Ng) such that, in analysis of proliferation, inhibition mediated by Nm OMVs was dependent on prior lymphocyte activation, whereas challenge with CEACAM1 reactive Ng OMVs inhibited proliferation in the absence of lymphocyte prestimulation. We also consider it noteworthy that Nl OMVs (selected according to the invention to be Opa-free) did not induce lymphocyte proliferation while Opa-ve (or otherwise CEACAM1 non-reactive) Ng OMVs induced proliferation relative to unchallenged lymphocytes.

The invention also differs surprisingly from a previous study in which infection with intact bacteria (expressing Opa) had little appreciable effect on cell death. In the invention, compositions prepared from those bacteria, e.g. OMvs, did affect cell death.

According to the invention, avoiding the use of CEACAM1 reactive Opa, (in the context of an OMV) inhibits associated functions and has significant benefits in protective efficacy of all meningococcal vaccines. Commercially available (meningococcal) OMV vaccines are prepared from clinical isolates and as such are likely to be Opa⁺in the overwhelming majority of cases. In addition, >95% of expressed meningococcal Opa variants are recognised by CEACAM1 and consequently, it is highly probable that current vaccine preparations induce immunosuppressive effects through ligation of CEACAM1. Following the invention, vaccine “parent strains” are screened for Opa expression, and in particular, for CEACAM1 reactive species. Analysis of this type enables selection of Opa(−) or otherwise CEACAM1 non-reactive bacterial isolates, and consequently, a relative enhancement in immune response to OMVs obtained therefrom.

Both the meningococcus and gonococcus are obligate human pathogens and, consistent with this, Opa variants do not recognise murine or other CEACAM1, as already mentioned. Consequently, CEACAM mediated immunosuppression would not be evident in the animal models typically used to assess vaccine efficacy. Opa is an important neisserial pathogenicity determinant, and exclusion of this species may unduly restrict the potential immunogenicity of an OMV based (or other) vaccine. As a result, in accordance with specific embodiments of the invention a native non-immunosuppressive Opa, or a mutant protein modified to this end, is included in an OMV-based composition. Given the genetic plasticity typical of the Neisseriacae, and the availability of a complete meningococcal genome sequence antigenic selection and/or genetic manipulation can be used to engineer an optimised OMv preparation derived from the pathogenic Neisseriacae. Further, given the comparatively innocuous nature of the commensal species N. lactamica coupled with observations of heterologous protection afforded by OMVs from this species and, potentially, the expression of novel heterologous antigens in this context non-CEACAM1 reactive (or otherwise Opa^{-ve}) N. lactamica is also suitable for an improved N. meningitidis vaccine.

The invention is now illustrated in the following examples, with reference to the accompanying drawings in which:

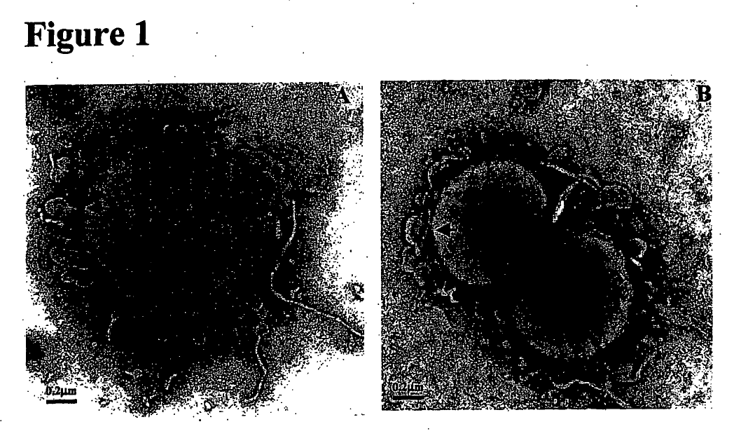

FIG. 1 is a scanning electron micrograph illustrating intact diplococci and isolated outer membrane vesicles (OMVs);

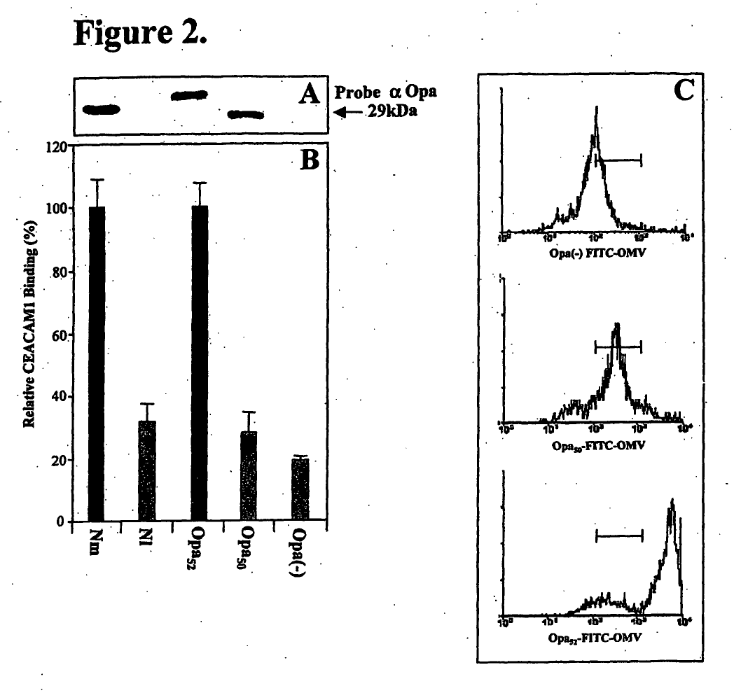

FIG. 2 shows Opa expression pattern and CEACAM1 binding properties of neisserial OMVs;

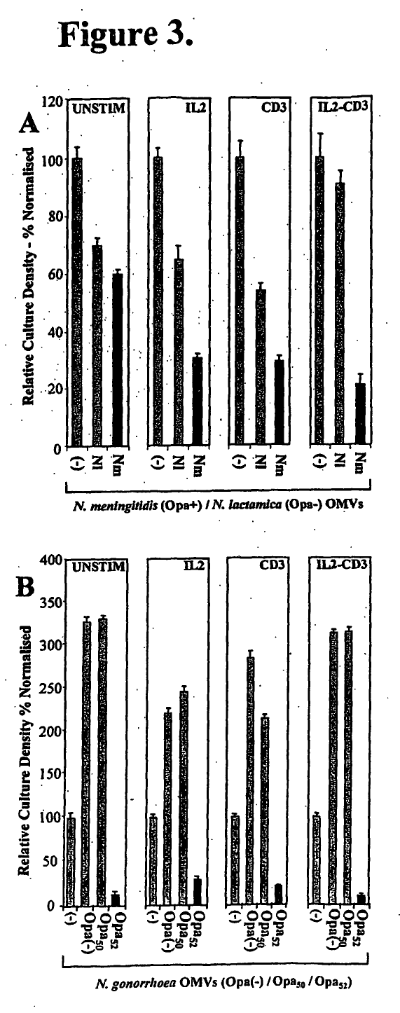

FIG. 3 shows proliferation of T lymphocytes in response to neisserial OMVs;

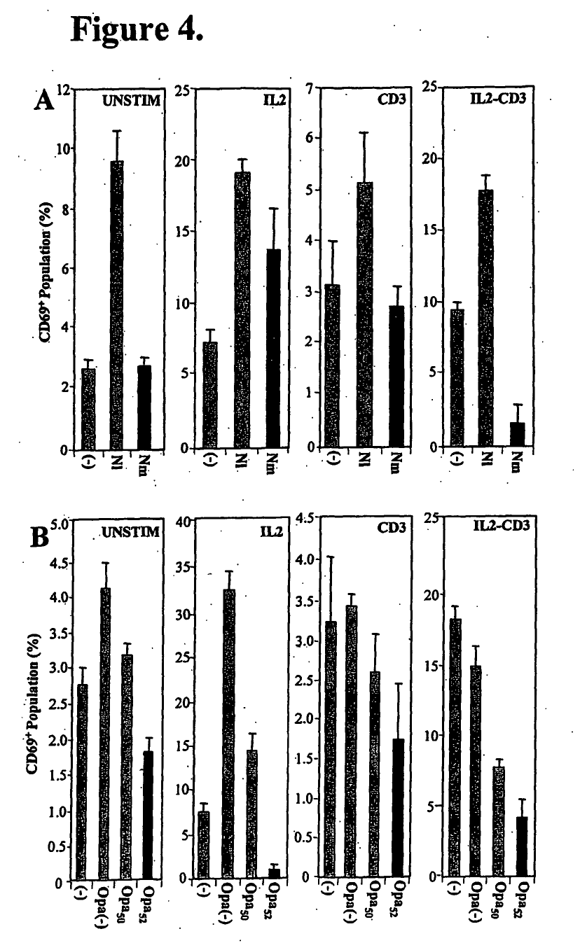

FIG. 4 is a FACS analysis of CD69 expression by Jurkat cells; and

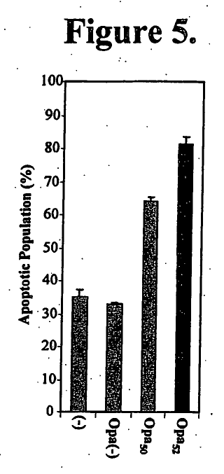

FIG. 5 shows apoptotic mortality among IL2 stimulated Jurkat cells in response 16 h challenge using gonococcal OMVs.

In more details, FIG. 1 shows a scanning electron micrograph illustrating intact diplococci and isolated outer membrane vesicles (OMVs). N. meningitidis (A), and N. lactamica (B) each have closely associated naturally occurring membrane “blebs” (filled arrows).

FIG. 2 shows Opa expression pattern and CEACAM1 binding properties of neisserial OMVs. (A) Immunoblot probed using_Opa protein specific mAb illustrating the presence of an immunoreactive Opa variant in Nm-OMVs, but not in comparable N1-OMVs; and appropriate Opa phenotypes in OMVs derived from isogenic gonococcal strains. (B): ELISA assay quantifying interactions between neisserial OMVs (10 μg total protein) and soluble CEACAM1-Fc. OMVs which recognize CEACAM1 are shown as black bars. In each instance, error bars indicate+1 SD (n=3).

FIG. 3 shows proliferation of T lymphocytes in response to neisserial OMVs. Jurkat T lymphocytes were cultured in the presence of 10,000 U/IL2 and/or 1 μg anti_CD3ε Ig and were then challenged using OMVs derived from either N. meningitidis (Nm), or N. lactamica (N1) (A); or N. gonorrhoeae expressing defined Opa variants (B). Challenge with CEACAM1 reactive OMVs are shown as black bars. In each instance, error bars indicate+1 SD (n=6). Statistical analysis of these data indicate that NL and NM data differ with a confidence interval of p<0.010 coincident with lymphocyte prestimulation and p=0.06 in the absence of prestimulation. Similar interrogation of gonococcal OMV data demonstrated that Opa₅₂ challenge data differed from comparable challenge data with a confidence interval of p<0.0002.

FIG. 4 is a FACS analysis of CD69 expression by Jurkat cells. Matched cell populations were prestimulated as indicated (using 10,000 U/IL2 and or 1 μg anti_CD3ε Ig) and were challenged with OMVs derived from N. meningitidis (Nm) or N. lactamica (Nl), (A), or using OMVs derived from N. gonorrhoeae, expressing defined Opa variants (B). In each instance, error bars indicate+1 SD (n=3 groups of 1×10⁵ events). Statistical analysis established that Nm and Nl data differed with a confidence interval ranging from p=0.01 to p<0.0001 with the most robust differences being evident either in unstimulated lymphocytes, or those prestimulated with IL-2 and anti_CD3ε Ig. Opa₅₂ challenge data differed from comparable data with confidence intervals ranging from p=0.0037−p<0.0001

FIG. 5 shows apoptotic mortality among IL2 stimulated Jurkat cells in response 16 h challenge using gonococcal OMVs. OMV challenge resulted in a dose dependent increase in apoptosis with the largest effects coincident with challenge using Opa52 OMV which react with CEACAM1. Challenge using Opa50 OMVs had an intermediate effect, and challenge using Opa(−) OMVs had a minimal effect on levels of apoptosis. In, each instance these data are representative of 1×10⁶ lymphocytes interrogated by flow cytometry. Data are representative of three experiments.

### EXAMPLE 1

Materials and Methods

Cell Lines and Tissue Culture Techniques

Jurkat (CD4⁺) human T lymphocytes (ATCC#CRL-10915) have been described previously {Nagasawa, Howatson, et al. 1981 ID:2798} and were routinely maintained in RPMI 1640 medium (Invitrogen; Burlington, Ontario) supplemented with 10% heat inactivated fetal bovine serum and 4 mM GlutaMAX (Invitrogen), referred to as RPMI-G. Cells were cultured at 37° C. in 5% CO₂ humidified air. Where appropriate, Jurkat cells were stimulated for 48 h using the indicated concentrations of recombinant human IL-2 (Pharmingen; Mississauga, Ontario) prior to OMV challenge. Challenges of this type were carried out in RPMI-G supplemented with 5 U/ml benzonase endonuclease (Sigma; Oakville, Ontario) and 5% (v/v) phosphate-buffered saline (pH 7.4; PBS) with 1 mM MgCl₂ and 0.5 mM CaCl₂ (referred to as PBS/Mg/Ca). In some instances stimulation via the T cell receptor was induced by exposure to the human CD3ε-specific monoclonal antibody UCHT1 (Pharmingen), which was subsequently cross-linked using Fab₂ fragments of sheep anti-mouse IgG (Sigma; 3 μg/ml).

COS-7 African Green Monkey kidney cells (ATCC#CLR-1651) were maintained in DMEM (Invitrogen) supplemented with 10% heat inactivated FBS (Cansera; Etobicoke, Ontario), 100 units/ml penicillin/streptomycin, 1 mm glutamax, 1 mM sodium pyruvate and 1 mM non-essential amino acids (Invitrogen) at 37° C. in 5% CO₂ humidified air.

Bacterial Strains

Neisseria meningitidis strain K454 and Neisseria lactamica strain Y92 1009 isolates were obtained from the Meningococcal Reference Unit, Manchester, UK. Gonococcal strains constitutively expressing specific Opa variants of N. gonorrhoeae strain MS11 have been described previously, and were generously supplied by Dr. T. F. Meyer (Max-Planck-Institut für Infektionsbiologie, Berlin, Germany). Opa variants were expressed in the background of MS11 strain N279 which does not express pilin and has a deletion in OpaC₃₀ locus encoding this strain's only HSPG receptor-specific Opa variant. N. meningitidis and N. lactamica were grown from frozen stocks on Mueller Hinton Agar (Difco Labs; West Molesey, Surrey, UK) and N. gonorrhoeae strains were grown from frozen stocks on GC agar (Difco; Oakville, Ontario), supplemented with 1% (v/v) Iso VitaleX™ enrichment (BBL™; Becton Dickinson, Cockeysville, Md.). All bacterial strains were cultured at 37° C. in 5% CO₂ humidified air and were sub cultured daily, using a binocular microscope to monitor Opa phenotype. Opa expression and variant-type were routinely confirmed by SDS-PAGE (10%) with resolved proteins either being stained using Coomassie Brilliant Blue or subjected to immunoblot analysis using the Opa specific monoclonal antibody 4B12/C11, which reacts with all known Opa variants and was generously provided by Dr. M. Achtman (Max-Planck-Insitut für Infektionsbiologie, Berlin, Germany).

Preparation and Physical Characterization of OMVs

OMVs were prepared from N. meningitidis and N. lactamica isolates. Overnight liquid cultures were prepared in Franz medium, and pelleted by centrifugation at 1000×g. Bacteria were resuspended in OMv buffer containing 0.15 M NaCl, 0.05 M Tris-HCl, 0.01 M EDTA (pH 7.5). Bacterial suspensions were then heated to 56° C. for 30 minutes, and resultant extracts then clarified by centrifugation at 25,000×g for 20 min. Recovered supernatants were centrifuged at ˜100,000×g for 2 h. The final OMV-containing pellet was washed twice, resuspended in PBS and stored at −80° C.

Gonococcal OMVs were prepared from recombinant gonococcal strains with defined Opa phenotypes. Bacteria were passaged overnight on solid medium (as described above), and near stationary phase liquid cultures were prepared in modified Brain Heart Infusion (Difco) containing 10 mM LiCl, 1 mM MgCl, 2 mM CaCl, 50 mM Hepes, 1% D-Glucose (pH 7.2), cultured at 37° C. in 5% CO₂ humidified air with rapid shaking. Thereafter, incubation was continued for an additional 2 h at 40° C. with rapid shaking. Bacteria were removed by centrifugation at 1000×g for 20 min, and resuspended in PBS containing 0.05% (w:v) sarkosyl (Bioshop Canada, Inc; Burlington, Ontario) and 0.05% sodium deoxycholate (w:v) (Bioshop Canada, Inc). Resuspended cells were incubated at 56° C. for 30 min. with gentle mixing and then chilled on ice. Bacterial suspensions were then extracted by using a Wheaton homogenizer and were then sonicated on ice (5×10 s pulses). Extracts were clarified by centrifugation at 25,000×g for 20 minutes, and the resulting supernatant centrifuged at 100,000×g for 2 h. The final pellet, which contains OMVs, was washed twice, resuspended in PBS, extruded through a 0.22 μm syringe filter, and stored at −80° C. OMV sterility was tested by inoculation onto GC agar and incubation as described above. The size distribution of OMVs was determined either by scanning electron microscopy (N. lactamica and N. meningitidis OMVs) by comparative FACS analysis (gonococcal OMVs). Estimates of relative surface area were calculated based on the relationship S=4πr² where S represents the surface area of a sphere, and r represents the radius of that sphere.

Construction and Expression of CEACAM1-Fc Fusion Proteins

An expression vector encoding the CEACAM1 amino-terminal-domain fused to the Fc portion of human IgG1 was generously provided by Drs. O. Mandelboim and G. Markel (Hadassah Medical School, Jerusalem, Israel). Recombinant CEACAM1-Fc protein was expressed in COS-7 cells were transiently transfected using FuGENE6 reagent (Roche Molecular Biochemicals, Indianapolis, Ind., USA) according to the manufacturer's specifications. Cell culture supernatant was harvested 48-72 h after transfections and was clarified by centrifugation at 1000×g for 20 minutes at 4° C. Clarified supernatant was then filtered using a vacuum-driven disposable filtration system (Stericup 0.22 μm, Millipore; Nepean, Ontario), and concentrated using a 10 kDa-cut-off polyethersulfone ultrafiltration concentrator (Millipore). The fusion protein was then purified using Protein A-Sepharose (Sigma). Elution from this matrix was performed using 0.2 M glycine/HCl (pH 2.5), with aliquots being recovered directly into collection tubes containing 100 μl 1 M Tris (pH 9.0) to neutralize the samples. Purified eluate was dialyzed against PBS at 4° C. and then concentrated to less than 1 mL with Ultrafree Biomax centrifugal filters (Millipore). The receptor function and specificity of CEACAM1-Fc fusions was assessed by association with isogenic gonococcal strains possessing specific Opa protein variants

Determination of Opa Receptor Specificity.

Interactions between Opa variants and CEACAM1 were characterised by ELISA. Initially, the protein content of each OMV preparation was determined using the BCA assay system (Pierce Chemical Company, Rockford, Ill.), and samples containing equal amount of total protein were immobilized on 96 well microtitre plates (Coming Corporation; Midland Mich., USA). Each OMV was applied in triplicate serial doubling dilutions, and then exposed to a standard concentration of the CEACAM1-Fc fusion protein. Bound protein was detected using protein A-onjugated horseradish peroxidase, and visualized using the OPD colorometric system (Sigma). OPD-associated signal was quantified by spectrophotometric analysis at 450 nm.

Cytometric Analyses.

Jurkat activation was assessed by quantifying expression of the well-characterized T cell activation marker CD69. Cells were infected and/or challenged as described above, and after 16 h samples were probed using anti-human CD69 monoclonal antibody (clone FN50) conjugated to allophycocyanin (Pharmacia; Mississauga, Ontario). Cells were then fixed in paraformaldehyde (3.7%) and CD69-associated fluorescence was assessed using a FACScalibur flow cytometer (Becton Dickinson, Oakville, Ontario). Cell death was characterized and quantified using the Annexin-V-FLUOS/Propidium iodide staining kit (Boehringer Mannheim, Laval, Quebec), thereby allowing proportional comparison of live, apoptotic and necrotic populations by flow cytometry.

Analysis Of Lymphocyte Proliferation.

Parallel, density-matched, cultures of Jurkat cells were challenged in RPMI-G+5% PBS/Mg/Ca using OMVs at the indicated concentration. Gentamycin (100 μg/ml; Bioshop) was added 2 h after the commencement of infection and was maintained throughout the subsequent incubation period to prevent bacterial overgrowth. Some cells were cultured in the presence of 10,00 U recombinant human IL-2 (Pharmingen) and stimulation via the T cell receptor was induced by exposure to 1 μg/ml mouse anti-human CD3ε IgG (clone UCHT1; Pharmingen). Jurkat proliferation was assessed by direct counting using a Levy double hemocytometer 72 h following infection. In each instance, proliferation was assessed using a standardized counting pattern and no less than six fields were counted for each sample.

Results

Opa protein presented by neisserial OMVs. Neisseria sp. naturally overproduce, evaginate and release outer membrane vesicles (OMVs) or “blebs” (FIG. 1A-B). OMVs obtained from N. meningitidis (Nm-OMV) and N. lactamica (Nl-OMV) are currently being assessed for efficacy as a vaccine providing protection against meningococcal disease. OMVs were also prepared from isogenic strains of N. gonorrhoeae expressing well-defined Opa variants, including the transparent (Opa⁻) strain N302, the heparan sulfate proteoglycan (HSPG) receptor-specific Opa₅₀-expressing N303, and the CEACAM-specific Opa₅₂-expressing N309. Gonococcal OMVs (Ng-OMV) were of similar size to those in the vaccine preparations, as determined by comparative flow cytometric analysis (data not shown).

Equivalent amounts of each OMV, as determined by protein content, were grossly characterized by SDS-PAGE (data not shown). Consistent with the phase and antigenic variability of neisserial outer membrane proteins {Nassif, Pujol, et al. 1999 ID: 2839}, considerable variation was apparent between the various preparations (data not shown). Immunoblots probed using the Opa-specific monoclonal antibody (mAb) that cross-reacts with all known Opa variants illustrated the presence of a single Opa variant in Nm-OMVs, while none was apparent in the Nl-OMV preparation (FIG. 2A). Appropriate Opa phenotypes were confirmed in OMVs derived from the recombinant gonococcal strains (FIG. 2A). The OMVs containing non-CEACAM1 reactive Opa and the Opa-free Nl-OMVs were therefore selected as suitable for the invention and were compared with Opa-containing Nm-OMVs. Where expressed, Opa variants were present in comparable concentrations, indicating no gross differences with regard to the density of Opa proteins per unit OMV protein.

Equal amounts of OMVs were exposed to soluble CEACAM1-Fc fusion proteins in an ELISA assay. This analysis indicated that the Opa⁺ Nm-OMVs, and those derived from Opa₅₂-expressing gonococci bound the soluble CEACAM1-Fc fusion at levels significantly (p<0.0001) greater than either the Nl-OMVs, which lack Opa proteins, or those derived from either transparent (Opa⁻) or Opa₅₀-expressing gonococci. This indicated that the CEACAM1-specific binding function of gonococcal Opa₅₂ was maintained in OMv preparations, and demonstrated that the Opa protein variant present in Nm-OMVs recognizes CEACAM1. Differences in receptor recognition remained significant with OMv protein concentrations ranging from 0.1 μg/ml to 10 μg/ml (FIG. 3 and data not shown).

CEACAM-specific OMVs inhibit lymphocyte activation. Previously, we have observed that gonococci expressing CEACAM1-specific Opa variants suppress CD4⁺ T cell proliferation in response to activating stimuli. Given that Nm-OMVs differed from Nl-OMVs with respect to Opa phenotype (FIG. 2A), and CEACAM1 binding (FIG. 2B), we tested whether there was a difference in lymphocyte response to these standard vaccine preparations. The degree of inhibitory effect was determined by the presence of a CEACAM1 binding Opa variant, and further, was influenced by the type of stimulation applied to the lymphocytes. A small difference in cell culture growth (p<0.06) was apparent when Nl-OMV and Nm-OMV were applied to unstimulated Jurkat CD4⁺ T cells. However, when lymphocytes were stimulated using the cytokine IL-2 and/or T cell receptor ligation prior to OMV challenge, the Opa associated differences increased in significance (p<0.01) (FIG. 3A). To confirm that CEACAM1 binding was sufficient to confer this inhibitory effect, we compared the effect of OMVs derived from isogenic gonococcal strains expressing defined Opa variants with distinct receptor specificities. In contrast to effects observed following challenge using the Nl- or Nm OMVs, challenge using either Opa⁻ or Opa₅₀-containing OMVs stimulated proliferation of primary CD4⁺ T lymphocytes. This is consistent with previous observations made using intact bacteria (rather than OMVs) as the challenge species. However, Opa₅₂-expressing OMVs potently suppressed Jurkat cell proliferation (FIG. 3B), confirming that this CEACAM1-specific Opa variant retains both its receptor binding and co-inhibitory functions in this context.

Reduced lymphocyte proliferation could result from a slower rate of cell division among the entire population and/or a reduction in the proportion of cells being stimulated to divide. To determine the proportion of cells that become activated in the presence of various OMV preparations, we quantified expression of the activation marker CD69 by Jurkat T cells. Parallel density-matched lymphocyte populations were pre-stimulated, and then challenged with various OMV preparations. Exposure to CEACAM1-reactive OMV s consistently reduced the proportion of lymphocytes expressing CD69 (FIGS. 4A and B). In the case of the two vaccine preparations, the number of activated Jurkat cells in response to Nm-OMVs was consistently lower than that observed with Nl-OMVs, and was either comparable to (unstimulated and anti-CD3ε-containing samples) or lower than (anti-CD3ε-containing samples) parallel samples not exposed to OMVs (FIG. 4A). Challenge with gonococcal OMVs (FIG. 4B) demonstrated a consistent pattern of cellular activation such that, in all conditions tested, the presence of OMVs containing the CEACAM-specific Opa₅₂ was coincident with reduced cellular activation. However, in this instance, levels of activation were marginally reduced by exposure to Opa⁺ OMVs (irrespective of receptor specificity) but were, in all instances, more significantly inhibited by exposure to CEACAM1 reactive OMVs (p<0.001).

Neisserial OMVs induce apoptosis in Jurkat cells. To quantify and directly compare the influence of Opa protein expression on cell death, we monitored apoptosis and necrosis in Jurkat cell cultures exposed to various dilutions of gonococcal OMVs (FIG. 5). While cellular necrosis was consistently below 5% irrespective of challenge conditions (data not shown), we observed a dose-dependent increase in apoptosis in response to all three OMV preparations. Apoptosis was clearly higher in response to OMVs containing Opa proteins, suggesting that the bulk of cell death correlated with attachment of OMVs to the lymphocyte surface. Opa₅₂-containing OMVs induced greater apoptosis than those containing Opa₅₀, indicating that some part of this effect was dictated by receptor-specific effects. These results contrast those in which intact gonococci were used as the challenge species, as we have observed no significant induction of either apoptosis or necrosis in response to infection by transparent (Opa⁻), Opa₅₀- or Opa₅₂-expressing bacteria.

### EXAMPLE 2

EMS Mutagenesis Protocol

The protocol is as follows:

Grow N.meningitidis to an optical density at 600 nm (OD₆₀₀) of 0.7 in Franz medium. Transfer 6 ml to a 15-ml conical tube; spin down for 5 min at 2,000 g.

Wash the cells twice with 10 ml of ethyl methanesulfonate (EMS) mutagenesis buffer.

Resuspend the washed cell pellet in 12 ml of the EMS mutagenesis buffer.

Dispense the cell suspension in four 2.4 ml aliquots into 15-ml conical tubes. In a fumehood, add 62.5 μl of EMS (Sigma catalog number M0880) to each aliquot of prepared cell suspension and vortex thoroughly (EMS takes a while to go into solution).

Incubate samples for 0, 1, 1.25 and 1.5 h at 37° C., which should give roughly 100, 80, 50 and 20% survival respectively.

To process samples add 10 ml of EMS stop solution; spin down the cells: wash once with 10 ml of EMS stop solution; wash the cells once with 10 ml of medium; resuspend the washed pellet in 2.5 ml of medium and store the cell suspension at 4° C.

To determine the viable-cell titer of mutagenized samples, sonicate each sample five times with a Braun Sonic U sonicator set at B070. Serially dilute and plate in triplicate on selective medium. Incubate plates at 37° C.

To plate out mutagenized cells for single colonies, filter each sonicated sample though 5 mm pore filters. The filtration step is necessary in order to obtain a good single cell suspension; however about 95% of total cells are lost during filtration due to removal of clumps of cells. Serially dilute filtered samples and plate on selective medium. Incubate plates at 37° C.

Screen the colonies for bacteria which express CEACAM1-non reactive Opa proteins. Subsequently screen for such Opa proteins that induce antibodies which bind native, CEACAM1-reactive Opa.

EMS Mutagenesis Buffer

4.23 ml 1M NaH₂PO₄
5.77 ml 1M Na₂HPO₄
0.5 ml 20% Tween 80
H₂O to 100 ml
   EMS Stop Solution
5% sodium thiosulfate, 0.1% Tween 80, filter sterilized, 100 ml (make fresh)

### EXAMPLE 3

NTG Mutagenesis

The protocol is as follows:

Grow N. meningitidis to an optical density at 600 nm (OD₆₀₀) of 0.8 in Franz medium. Transfer 50 ml to a 50-ml conical tube; spin down for 15 min at 2,000 g.

Discard the supernatant and resuspend the pellet in 5 ml of medium.

Aliquot 1 ml of cells to individual 15 ml round-bottom tubes (Falcon cat. No. 2059) and add nitrosoguanidine (NTG) to a final concentration range of 0 to 1,000 mg/ml.

Incubate the cell suspension at 37° C. with shaking for 1 h.

Wash each aliquot of cells twice with 10 ml of medium and finally resuspend the pellet in 1 ml of medium.

Make serial dilutions of each reaction and plate in duplicate to get colony counts for determination of the killing curve.

Repeat the mutagenesis with the concentration of NTG that yields 90% killing in the above conditions.

Screen the colonies for bacteria which express CEACAM1-non reactive Opa proteins. Subsequently screen for such Opa proteins that induce antibodies which bind native, CEACAM1-reactive Opa.

### EXAMPLE 4

Generation of an Opa knock-out Mutant

Multiple technologies exist by which to mutate expression of Opa protein variants from the three or four Opa alleles present in N. meningitidis (or similar alleles in other Neisseria and/or other bacteria). Briefly, Opa alleles are cloned by virtue of highly conserved regions, in non-coding regions adjacent to regions encoding the Opa peptide itself. Genes cloned in this way are inserted into a suitable plasmid vector and mutagenised using either transposon insertion, or other standard molecular biology techniques (suitable techniques are set out in Sambrook, Fritsch and Maniatis, 1989, Comelissen et al, 1992; Comelissen and Sparling 1996, and Boulton et al, 2000).

### EXAMPLE 5

Generation of a Fragment of Opa that Does not Bind to CEACAM1

Opa is expressed either in the context of a native organism (ie a Neisseria sp) or a single, cloned, recombinant Opa variant is over-expressed in a heterologous context. In either instance, intact Opa is purified either from bacterial whole cell lysates, purified bacterial membranes or from inclusion bodies, using standard biochemical and/or biophysical techniques, including affinity chromatography and/or selecting filtration and/or gel infiltration chromatography and/or ion exchange chromatography.

Purified Opa is then fractionated by proteolytic digestion using trypsin, chymotrpsin, subtilisin and/or other proteolytic enzymes and/or other chemical treatments. Opa fragments produced in this way are selected for an absence of CEACAM1 binding, by affinity chromatography, using the CEACAM1 N terminal—Fc fusion protein described herein, (i.e., by selection of protein fragments which fail to bind this fusion protein). Fragments of this type are used as an antigen in a suitable animal system, in the presence of an adjuvant preparation, and optionally conjugated to a suitable carrier protein. Serum prepared in this way is tested against intact Opa variants by Western blot analysis, and/or whole cell dot blots, and/or cytometric analysis, and/or other reactivity assays. Alternatively, an array of overlapping Opa specific peptides is synthesised de novo using standard techniques (Gausepohl et al, 1992), or otherwise expressed from cloned, recombinant nucleic acid sequences encoding specific regions of Opa. In either instance, peptides generated (using either or both techniques) are screened for CEACAM1 binding according to methods we have defined previously, and can, subsequently, be used as antigens as described.

### EXAMPLE 6

Generation of Non-CEACAM1 Reactive Opa Variants

Several naturally occurring gonococcal Opa variants have no CEACAM1 binding activity, (Gray-Owen et al., 1997) and have been evaluated in terms of immunomodulatory effects. It is also established that, of the four naturally occurring meningococcal Opa variants at least one variant is similarly devoid of CEACAM1 binding activity (Meutmer et al, 2000), and, in addition, commensal Neisseria sp. express Opa variants which do not recognize CEACAM1 (Toleman, Aho and Virji, 2001).

A given bacterial culture is screened for Opa expression and CEACAM1 reactivity (or its absence), and the results used to select a phenotype that is non CEACAM1 reactive. Opa expression is phase variable, and therefore it is preferred that this process of selection be performed on a routine basis. The screening is suitably performed prior to OMV purification. It can also be performed on OMV preparations from Gram negative bacteria.

Alternatively, a bacterium (preferably a neisserial strain) is constructed that is capable of expressing a single Opa of defined phenotype and receptor specificity. This is achieved by replacing a non functional (i.e. mutated) Opa allele, in the bacterial chromosome with a native (i.e. non-mutated) sequence. Alternatively, Opa is cloned in series with one of several well characterized bacterial gene regulation systems; Opa expression is then controlled by manipulation of culture conditions.

The invention thus provides microorganisms, compositions, vaccines, components of vaccines, methods of obtaining the aforementioned and genes encoding the aforementioned, substantially free of Opa that binds CEACAM1. These are suitable for use in treatment or prevention of disease caused by Gram negative bacteria, especially Neisseria

1-43. (canceled)

44. A method of treatment or prevention of meningococcal disease comprising administering to a subject an effective amount of Neisseria outer membrane vesicles which contain Opa that does not bind to CEACAM1 which are substantially free of Opa that binds CEACAM1, wherein said outer membrane vesicles are from Neisseria that have been modified by mutation to express an Opa that does not bind to CEACAM1.

45. The method of claim 44, wherein activation or proliferation of CD4+ T cells is enhanced.

46. The method of claim 44, wherein said Neisseria is Neisseria meningitidis.

47. The method of claim 44, wherein stimulation of immune memory is improved or inhibition of T cell function is reduced.

48. The method of claim 44, wherein said mutation is by a method mutagenesis selected from the group consisting of transposon mutagenesis, UV light, EMS mutagenesis and NTG mutagenesis.

49. The method of claim 44, wherein said administering is selected from the group consisting of parenteral, intramuscular, trans-dermal, intra-nasal, oral, topical or mucosal.

50. The method of claim 44, wherein said outer membrane vesicles comprise a heterologous antigen.

51. A method of treatment or prevention of meningococcal disease comprising administering to a subject an effective amount of a composition comprising Neisseria outer membrane vesicles, wherein said outer membrane vesicles are substantially free of Opa that binds to CEACAM1.

52. The method of claim 51, wherein stimulation of immune memory is improved or inhibition of T cell function is reduced.

53. The method of claim 51, wherein said composition comprises a carrier.

54. The method of claim 53, wherein said carrier is selected from the group consisting of saline solution, sucrose solution, or a pharmaceutically acceptable buffer solution.

55. The method of claim 51, wherein said composition comprises a surfactant.

56. The method of claim 51, wherein said composition comprises an adjuvant.

57. The method of claim 51, wherein said composition comprises microencapsulated outer membrane vesicles.

58. The method of claim 57, wherein said microencapsulated outer membrane vesicles comprise a biocompatible polymer shell or core.

59. The method of claim 58, wherein said biocompatible polymer shell or core is made from polylactide-co-glycolide.

59. A method of preparing a vaccine composition for treatment or prevention of meningococcal disease, the method comprising:
(a) isolating Neisseria outer membrane vesicles which contain Opa that does not bind to CEACAM1 and which are substantially free of Opa that binds CEACAM1, wherein said outer membrane vesicles are from Neisseria that have been modified by mutation to express an Opa that does not bind to CEACAM1; and
(b) formulating the composition for use as a vaccine.

60. A method of preparing a vaccine composition for treatment or prevention of meningococcal disease, the method comprising:
(a) obtaining a Neisseria;
(b) determining whether the Neisseria expresses an Opa protein that binds to CEACAM1;
(c) if the Neisseria expresses an Opa protein that binds to CEACAM1, discarding the Neisseria and repeating steps (a) to (c);
(d) retaining the Neisseria if it expresses a mutant or variant or fragment or derivative of Opa, wherein the mutant or variant or fragment or derivative does not bind to CEACAM1; and
(e) preparing a composition comprising the retained Neisseria of step (d).

61. The method of claim 60, wherein said mutant or variant or fragment or derivative is obtained by:
(i) obtaining a Neisseria;
(ii) carrying out mutagenesis on the Neisseria;
(iii) determining whether the Neisseria expresses a mutant or fragment or variant or derivative of an Opa protein that does not bind to CEACAM1;
(iv) isolating said mutant or variant or fragment or derivative, wherein the mutant or variant or fragment or derivative does not bind to CEACAM1.

62. The method of claim 61, wherein said mutagenesis is selected from the group consisting of transposon mutagenesis, UV light, EMS mutagenesis and NTG mutagenesis.

63. The method of claim 60, wherein said determining comprises exposing said Opa protein to a CEACAM1-Fc fusion protein in an ELISA assay.

64. The method of claim 63, wherein said determining further comprises contacting said Opa protein with an Opa-specific monoclonal antibody.

65. The method of claim 60, wherein said determining comprises characterizing the interaction between said Opa protein and CEACAM1 by ELISA.

66. The method of claim 61, further comprising:
(v) raising an antibody to the mutant or fragment or variant or derivative; and
(vi) determining whether the antibody also binds to an Opa protein that binds to CEACAM1.

67. The method of claim 60, wherein the Neisseria is Neisseria meningitidis.

68. The method of claim 60, comprising preparing an outer membrane vesicle from the retained Neisseria.
